(19) United States
(12) Patent Application Publication
(10) Pub. No.: US 2025/0054205 A1
(43) Pub. Date: Feb. 13, 2025

(54) Method And System For Reconstructing Image Of Midpalatal Suture Based On Sobel Operator

(71) Applicants: Beijing Children's Hospital, Capital Medical University, Beijing (CN); Dfusion (Beijing) Technology Co., Ltd., Beijing (CN)

(72) Inventors: Lu Gao, Beijing (CN); Guoxia Yu, Beijing (CN); Zhiyu Chen, Beijing (CN); Yi Lei, Beijing (CN); Qing Wang, Beijing (CN)

(21) Appl. No.: 18/232,933

(22) Filed: Aug. 11, 2023

(51) Int. Cl. G06T 11/00; A61B 6/00; G06T 5/50; G06T 7/40; G06T 7/60

(52) U.S. Cl. CPC G06T 11/005; A61B 6/501; A61B 6/505; G06T 5/50; G06T 7/40; G06T 7/60; G06T 11/006; G06T 2207/10081; G06T 2207/20048; G06T 2207/20221; G06T 2207/30036; G06T 2210/41; G06T 2211/40

(57) ABSTRACT

The present disclosure relates to a method and system for reconstructing an image of a midpalatal suture based on a Sobel operator and belongs to the technical field of image processing. Firstly, data preprocessing is performed on a cone beam computed tomography (CBCT) file of a midpalatal suture of a maxilla to obtain a plurality of local images of the midpalatal suture. Merging fusion is then performed on the plurality of local images of the midpalatal suture to obtain a reconstructed image of the midpalatal suture, and a Sobel operator is utilized for optimization during the merging fusion. Thus, an overall profile of a region shape of the midpalatal suture can be reconstructed in a same image from midpalatal suture regions distributed in multiple layers of CBCT images, facilitating intuitive and accurate judgment on the midpalatal suture region by a doctor.

### TECHNICAL FIELD

The present disclosure relates to the technical field of image processing, and in particular, to a method and system for reconstructing an image of a midpalatal suture based on an optimized Sobel operator.

### BACKGROUND

The midpalatal suture of a maxilla, which refers to a suture located between horizontal plates of palatine bones on two sides, can be utilized to detect the development of a maxillary width of a teenager and evaluate the maxillary development potential of the teenager. However, the midpalatal suture of the maxilla is present as an arched region. The midpalatal suture can be observed by directly viewing tomographic images displayed by a cone beam computed tomography (CBCT) file. Since each image can only display a portion of the arched midpalatal suture, a doctor cannot intuitively feel or observe the overall profile of the midpalatal suture and thus can hardly accurately judge the development of the maxillary width of a teenager based on the situation of the midpalatal suture.

On this basis, there is an urgent need for a method and system for observing the overall profile of the midpalatal suture region in an image.

### SUMMARY

An objective of the present disclosure is to provide a method and system for reconstructing an image of a midpalatal suture based on a Sobel operator that can reconstruct an overall profile of a region shape of a midpalatal suture in a same image from midpalatal suture regions distributed in multiple layers of CBCT images, facilitating intuitive and accurate judgment on the midpalatal suture region by a doctor.

To achieve the above objective, the present disclosure provides the following technical solutions.

A method for reconstructing an image of a midpalatal suture based on a Sobel operator includes the following steps:
performing data preprocessing on a cone beam computed tomography (CBCT) file of a midpalatal suture of a maxilla to obtain a plurality of local images of the midpalatal suture; and
performing merging fusion on the plurality of local images of the midpalatal suture to obtain a reconstructed image of the midpalatal suture, where a Sobel operator is utilized for optimization during the merging fusion.

A system for reconstructing an image of a midpalatal suture based on a Sobel operator includes:
a data preprocessing module configured to perform data preprocessing on a CBCT file of a midpalatal suture of a maxilla to obtain a plurality of local images of the midpalatal suture; and
a fusing module configured to perform merging fusion on the plurality of local images of the midpalatal suture to obtain a reconstructed image of the midpalatal suture, where a Sobel operator is utilized for optimization during the merging fusion.

According to specific embodiments provided in the present disclosure, the present disclosure has the following technical effects:

The present disclosure is intended to provide a method and system for reconstructing an image of a midpalatal suture based on a Sobel operator. Firstly, data preprocessing is performed on a CBCT file of a midpalatal suture of a maxilla to obtain a plurality of local images of the midpalatal suture. Merging fusion is then performed on the plurality of local images of the midpalatal suture to obtain a reconstructed image of the midpalatal suture, and a Sobel operator is utilized for optimization during the merging fusion. Thus, an overall profile of a region shape of the midpalatal suture can be reconstructed in a same image from midpalatal suture regions distributed in multiple layers of CBCT images, facilitating intuitive and accurate judgment on the midpalatal suture region by a doctor.

### BRIEF DESCRIPTION OF THE DRAWINGS

To describe the technical solutions in the embodiments of the present disclosure or in the prior art more clearly, the accompanying drawings needing to be used in the embodiments will be briefly described below. Apparently, the accompanying drawings in the following description are merely some embodiments of the present disclosure, and for a person of ordinary skill in the art, other drawings may also be derived from these accompanying drawings without creative efforts.

FIG. 1 is a schematic diagram of a midpalatal suture provided by Example 1 of the present disclosure;

FIG. 2 is a flowchart of a method for reconstruction provided by Example 1 of the present disclosure;

FIG. 3 is a principle diagram of the method for reconstruction provided by Example 1 of the present disclosure;

FIG. 4 is a diagram illustrating comparison on effects of multiple methods for reconstruction provided by Example 1 of the present disclosure; and

FIG. 5 is a block diagram of a system for reconstruction provided by Example 2 of the present disclosure.

### DETAILED DESCRIPTION OF THE EMBODIMENTS

The technical solutions in the embodiments of the present disclosure will be clearly and completely described below with reference to the accompanying drawings in the embodiments of the present disclosure. Apparently, the described embodiments are merely a part rather than all the embodiments of the present disclosure. All other embodiments derived from the embodiments in the present disclosure by a person of ordinary skill in the art without creative efforts shall fall within the protection scope of the present disclosure.

An objective of the present disclosure is to provide a method and system for reconstructing an image of a midpalatal suture based on a Sobel operator that, by performing image extraction, fusion, and optimization on a CBCT file of a midpalatal suture of a maxilla, can reconstruct an overall profile of a region shape of the midpalatal suture in a same image from midpalatal suture regions distributed in multiple layers of CBCT images, facilitating intuitive and accurate judgment on the midpalatal suture region by a doctor.

To make the above-mentioned objective, features, and advantages of the present disclosure clearer and more comprehensible, the present disclosure will be further described in detail below with reference to the accompanying drawings and specific embodiments.

### Example 1

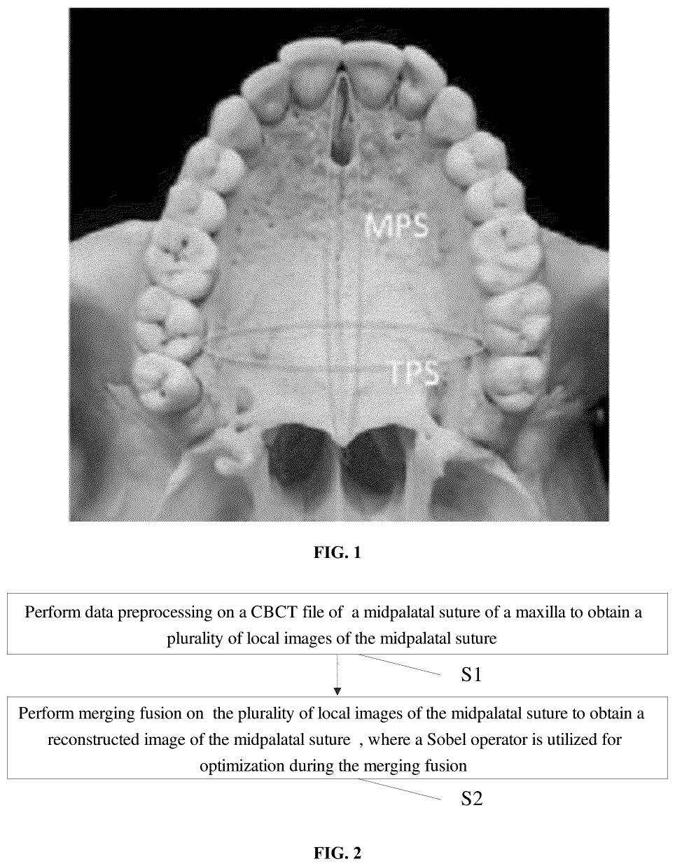

As shown in FIG. 1, a midpalatal suture is an arched region, and the overall profile of the arched midpalatal suture cannot be displayed in one image. To display the overall profile of the arched midpalatal suture in one image as much as possible, an existing approach utilized is as follows: when capturing medical CT images, the midpalatal suture is photographed obliquely at a certain angle in an extension direction thereof to obtain a series of oblique CBCT images, and an oblique image which is closest to the angle of the midpalatal suture and contains the maximum midpalatal suture region is picked therefrom for the purpose of restoring the overall profile of the midpalatal suture region to an utmost extent. However, such an approach may cause two problems: (1) it is difficult to find out a photographing angle: due to differences of human bodies, the midpalatal suture region of each person has a different camber and a different inclination degree, and therefore, before capturing oblique images, it is difficult to confirm what photographing angle should be utilized to restore the overall morphological profile of the midpalatal suture region to an utmost extent. (2) It is difficult to restore the overall profile: since the midpalatal suture region of the maxilla is an arched region, for a person having a small camber, oblique images can indeed present the overall morphological profile of the midpalatal suture to a certain extent, and for a person having a large camber of the midpalatal suture, oblique images can only reflect the morphology of part of the midpalatal suture region and cannot present the overall profile of the midpalatal suture region. On the whole, this approach has the disadvantages of low practicability and poor universality and thus cannot be promoted and utilized extensively.

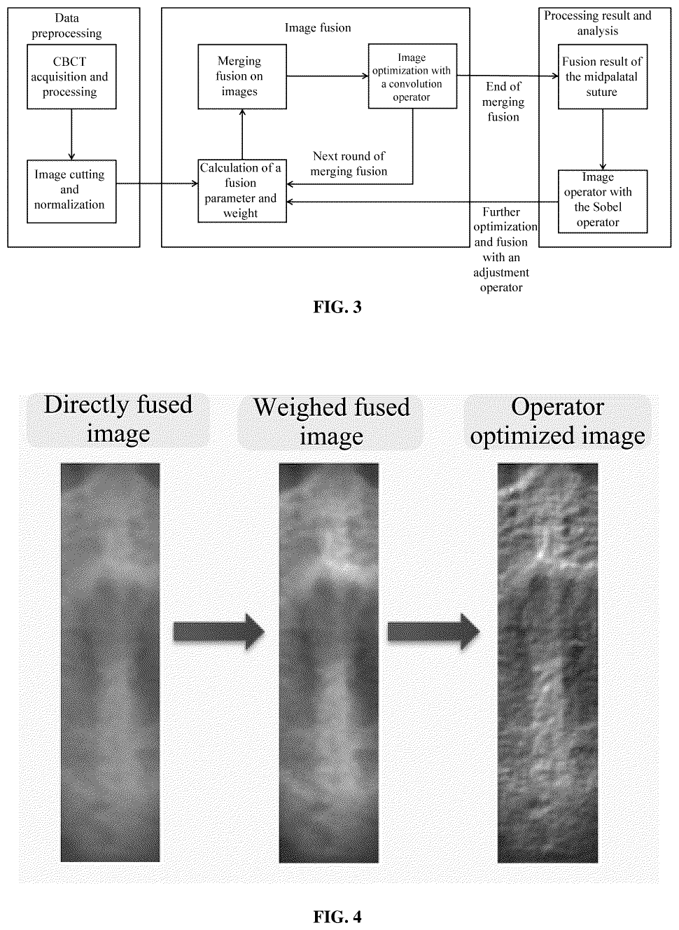

With the development of artificial development, it is possible and necessary to realize a technique for reconstructing a midpalatal suture based on a Sobel operator technique. An approach for fusing an image of a midpalatal suture region based on conventional CBCT images has the characteristics of ease of implementation and good universality and has promising application prospects. On this basis, an automatic and intelligent high-accuracy technique for reconstructing an image of a midpalatal suture is designed in combination with the expert knowledge in the art in the present example, and a method for reconstructing an image of a midpalatal suture based on a Sobel operator is provided, as shown in FIG. 2 and FIG. 3, including the following steps.

S1: data preprocessing is performed on a CBCT file of a midpalatal suture of a maxilla to obtain a plurality of local images of the midpalatal suture.

The CBCT file of the midpalatal suture of the maxilla can be directly acquired from a hospital database. Step S1 may include the following steps.

(1) The CBCT file of the midpalatal suture of the maxilla is read to obtain file information, where the file information includes a resolution, a number of layers, a window width, and a window level of the CBCT file of the midpalatal suture of the maxilla.

(2) The CBCT file of the midpalatal suture of the maxilla is transformed into a three-dimensional gray-level matrix based on the file information, and the three-dimensional gray-level matrix is transformed into a plurality of axial CT cross-sectional images.

The resolution and the number of layers determine a three-dimensional shape size of the three-dimensional gray-level matrix, and the window width and the window level determine a gray level interval of the three-dimensional gray-level matrix. In the present example, the transforming the CBCT file of the midpalatal suture of the maxilla into a three-dimensional gray-level matrix based on the file information may include the following steps: determine a three-dimensional size of the three-dimensional gray-level matrix based on the resolution and the number of layers; determine a gray level interval of the three-dimensional gray-level matrix based on the window width and the window level, and then transform the CBCT file of the midpalatal suture of the maxilla into the three-dimensional gray-level matrix based on the three-dimensional size and the gray level interval. For example, the resolution and the number of lavers determine a structure of the three-dimensional gray-level matrix. For example, given the resolution of 512*512 and 400 layers, the three-dimensional size of the three-dimensional gray-level matrix is 512*512*400. The window width and the window level are terms of medical CT. The gray level interval of the three-dimensional gray-level matrix may be obtained by simply computing the window width and the window level. The gray level interval will determine which tissues (e.g., blood vessels, viscera, or bones) in the image are more obvious.

The transforming the three-dimensional gray-level matrix into a plurality of axial CT cross-sectional images may include the following steps: transforming the three-dimensional gray-level matrix into a series of axial CT cross-sectional images by slicing in a height direction, and uniformly transforming the axial CT cross-sectional images into the resolution of 512*512. The axial CT cross-sectional images are images parallel to the plane determined by the x and y axes.

(3) Each of the axial CT cross-sectional images containing midpalatal suture regions is cut to obtain a plurality of local images of the midpalatal suture.

All the axial CT cross-sectional images are identified to extract the axial CT cross-sectional images containing midpalatal suture regions, and each of the axial CT cross-sectional images containing midpalatal suture regions is cut to obtain a plurality of local images of the midpalatal suture After the completion of cutting, the plurality of local images of the midpalatal suture are normalized and thus uniformly transformed into the resolution of 50*200.

S2: merging fusion is performed on the plurality of local images of the midpalatal suture to obtain a reconstructed image of the midpalatal suture, where a Sobel operator is utilized for optimization during the merging fusion.

The approach of fusing the images of the midpalatal suture regions based on a convolution operator used in the present example is the product from communicating with experts and optimizing an existing pixel-level image fusion algorithm. A fusion weight is mainly adjusted by combining the existing pixel-level image fusion algorithm with characteristics of the midpalatal suture regions, and the Sobel operator is utilized for optimizing the fused image, finally obtaining a reconstructed image of the midpalatal suture having a high resolution and obvious texture. Step S2 of image fusion process may include the following steps.

(1) The plurality of local images of the midpalatal suture are combined in pairs to obtain a plurality of image pairs to be fused, where pixels of the local images of the midpalatal suture included in each image pair to be fused correspond to one another one to one, and two pixels in the one-to-one correspondence are denoted as a pixel pair.

The plurality of local images of the midpalatal suture are combined in pairs in the following way: two local images of the midpalatal suture are randomly selected without repetition as an image pair to be fused. If the number of the local images of the midpalatal suture is an odd number, the remaining local image of the midpalatal suture is used as an image pair to be fused alone. For example, the number of the local images of the midpalatal suture is 5, the first local image of the midpalatal suture and the second local image of the midpalatal suture are used as an image pair to be fused, and the third local image of the midpalatal suture and the fifth local image of the midpalatal suture as an image pair to be fused, and the fourth local image of the midpalatal suture as an image pair to be fused, and three image pairs to be fused are obtained. When an image pair includes two local images of the midpalatal suture, pixels of the two local images of the midpalatal suture are in one-to-one correspondence with each other, and two pixels in the one-to-one correspondence are denoted as a pixel pair. When the image pair includes only one local image of the midpalatal suture, pixels of the local image of the midpalatal suture are denoted as a pixel pair.

Given N local images of the midpalatal suture, the number of the image pairs to be fused obtained by combining the plurality of local images of the midpalatal suture in pairs is: ┌(N+1)/2┐, where ┌ ┐ represents an operator of rounding down. If N=4, the resulting number obtained by combining in pairs is: ┌2.5┐=2; and if N=5, the resulting number obtained by combining in pairs is: ┌3┐=3.

(2) For each image pair to be fused, a fusion weight for each pixel pair is calculated, and merging fusion is performed on the image pair to be fused based on the fusion weight for each pixel pair to obtain a fused image.

The calculating a fusion weight for each pixel pair may include the following steps: calculate an overall average gray level and an adjustment factor based on gray level values of the plurality of local images of the midpalatal suture. i.e., calculate an average value of gray level values of all pixels of the plurality of local images of the midpalatal suture to obtain the overall average gray level, calculate a gray level difference between the gray level values of any two pixels of the plurality of local images of the midpalatal suture to determine a maximum gray level difference of the plurality of local images of the midpalatal suture, and determine the adjustment factor based on the maximum gray level difference; for each pixel pair, calculate an average value of gray level values of the image pair to be fused at the pixel pair to obtain an average gray level; and calculate the fusion weight for the pixel pair based on the overall average gray level, the adjustment factor, and the average gray level.

A calculation formula for the fusion weight for the pixel pair is as follows:

$${M}_{ij} = 1 + \frac{{A}_{ij} - e}{255 + d} ; \qquad ( 1 )$$

where Mᵢⱼ represents the fusion weight for pixel pair ij (i.e., the pixel pair composed of pixel i in one local image of the midpalatal suture and pixel j of the other local images of the midpalatal suture, and the position of the pixel i corresponds to that of the pixel j); Aᵢⱼ represents the average gray level of the pixel pair ij; e represents the overall average gray level; and d represents the adjustment factor. It needs to be noted that if the image pair to be fused includes only one local images of the midpalatal suture, the pixel pair ij is a single pixel, and in this case, the gray level value of the pixel is directly used as the average gray level Aᵢⱼ.

The performing merging fusion on the image pair to be fused based on the fusion weight for each pixel pair to obtain a fused image may include the following steps: for each pixel pair, calculate an average value of gray level values of the image pair to be fused at the pixel pair to obtain an average gray level; and calculate a fused gray level value of the pixel pair based on the average gray level and the fusion weight to obtain the fused image.

A calculation formula for the fused gray level value is as follows:

$${P}_{ij} = {A}_{ij} * {M}_{ij} = {A}_{ij} * ( 1 + \frac{{A}_{ij} - e}{255 + d} ) ; \qquad ( 2 )$$

where Pᵢⱼ represents the fused gray level value of the pixel pair ij.

After the fused gray level values of all the pixel pairs are obtained, the positions of all the pixel pairs remain unchanged. The fused image can be obtained by taking the fused gray level values as gray level values of the pixels at such positions.

In this example, the fusion weight for each pixel pair is calculated based on the gray level value of the pixel pair. The N local images of the midpalatal suture are then merged and combined in pairs based on the calculated fusion weights to obtain the fused images.

(3) Each fused image is optimized with the Sobel operator to obtain an optimized image.

The fused image is optimized with the Sobel operator such that the resulting optimized image has a more obvious high-frequency region and a higher overall contrast. The problems of image hazing and texture blurring caused by image fusion are avoided. Specifically, for each pixel of each fused image, an optimized gray level value of the pixel is calculated based on the Sobel operator to obtain the optimized image. After the optimized gray level values of all the pixels are obtained, the positions of all the pixels remain unchanged. The optimized image can be obtained by replacing the fused gray level values with the optimized gray level values.

(4) Whether a number of the optimized images is 1 is determined.

(5) If yes, the optimized image is taken as the reconstructed image of the midpalatal suture.

(6) If no, the optimized images are taken as the local images of the midpalatal suture in next loop, and the step of combining all local images of the midpalatal suture in pairs is carried out again.

Next merging fusion is performed on the optimized images. That is, steps (1) to (3) are repeated until the plurality of local images of the midpalatal suture are fused into one entire image of the midpalatal suture, thereby obtaining the reconstructed image of the midpalatal suture.

Here, the optimization principle and the implementation process of the Sobel operator are introduced.

The Sobel operators mainly utilized for edge detection and is a discrete difference operator for calculating an approximate gray level value of an image brightness function. The use of this operator at any pixel of an image will yield a corresponding gray level vector or a normal vector. This operator contains 8 different types of 3*3 matrices, i.e., in the following 8 directions: up, down, left, right, upper left, lower left, upper right, and lower right, respectively. Transverse and longitudinal approximate brightness difference values can be obtained by subjecting the operator and an image to planar convolution, respectively. If an original image is represented by A, Gₓ and G_{y} represent image gray level values resulting from transverse edge detection and longitudinal edge detection, respectively, and a formula for the planar convolution of the Sobel operator and the image is as follows:

$${G}_{x} = [ \begin{matrix}- 1 & 0 & + 1 \\ - 2 & 0 & + 2 \\ - 1 & 0 & + 1\end{matrix} ] * A , {G}_{y} = [ \begin{matrix}+ 1 & + 2 & + 1 \\ 0 & 0 & 0 \\ - 1 & - 2 & - 1\end{matrix} ] * A ; \qquad ( 3 )$$

The two matrices in the formula (3) are the Sobel operator.

Their specific calculation formulas are as follows:

$${G}_{x} = ( - 1 ) * f ( x - 1 , y - 1 ) + 0 * f ( x , y - 1 ) + 1 * f ( x + 1 , y - 1 ) + ( - 2 ) * f ( x - 1 , y ) + 0 * f ( x , y ) + 2 * f ( x + 1 , y ) + ( - 1 ) * f ( x - 1 , y + 1 ) + 0 * f ( x , y + 1 ) + 1 * f ( x + 1 , y + 1 ) = [ f ( x + 1 , y - 1 ) + 2 * f ( x + 1 , y ) + f ( x + 1 , y + 1 ) ] - [ f ( x - 1 , y - 1 ) + 2 * f ( x - 1 , y ) + f ( x - 1 , y + 1 ) ]$$

$${G}_{y} = 1 * f ( x - 1 , y - 1 ) + 2 * f ( x , y - 1 ) + 1 * f ( x + 1 , y - 1 ) + 0 * f ( x - 1 , y ) + 0 * f ( x , y ) + 0 * f ( x + 1 , y ) + ( - 1 ) * f ( x - 1 , y + 1 ) + ( - 2 ) * f ( x , y + 1 ) + ( - 1 ) * f ( x + 1 , y + 1 ) = [ f ( x - 1 , y - 1 ) + 2 f ( x , y - 1 ) + f ( x + 1 , y - 1 ) ] - [ f ( x - 1 , y + 1 ) + 2 * f ( x , y + 1 ) + f ( x + 1 , y + 1 ) ]$$

where f(a,b) represents the gray level value of pixel (a,b) of the image.

The image gray level values resulting from transverse edge detection and longitudinal edge detection of each pixel (x,y) of the image are combined through the following formula to calculate the gray level value of the pixel (x,y):

$$G = \sqrt{{G}_{x}^{2} + {G}_{y}^{2}} ; \qquad ( 4 )$$

If the gray level value G is greater than a threshold, the pixel (x,y) is considered to be an edge pixel.

In terms of image optimization, the Sobel operator with an appropriate weight is directly utilized in the present example to convolute the image so that the edge of the image can be sharpened to a certain degree and the texture can be enhanced Specifically, in image processing, a basic processing approach is linear filtering: a planar digital image to be processed may be regarded as a large matrix, and each pixel of the planar digital image to be processed corresponds to each element of the large matrix; a small filter matrix (i.e., a convolution operator) is utilized for filtering; the small filter matrix is a square matrix, i.e., having rows as many as columns; the purpose of filtering is to, for each pixel in the large matrix, calculate products of neighboring pixels thereof and the elements of the small filter matrix at the corresponding positions, then summing the products, and using the final resulting value as a new value for the pixel. Thus, one round of filtering is completed. In the present example, the fused image is regarded as a large matrix, and each pixel of the fused image corresponds to each element of the large matrix. The Sobel operator is regarded as the small filter matrix for filtering. For each pixel in the fused image, the optimized gray level value G of the pixel is calculated by the formula (3) and the formula (4). The positions of all the pixels remain unchanged, and the optimized image can be obtained by replacing the fused gray level values with the optimized gray level value G.

As an alternative embodiment, after a round of fusion is completed, i.e., after the reconstructed image of the midpalatal suture is completed through a round of complete merging, the texture feature of the reconstructed image of the midpalatal suture is analyzed, and the weight of the Sobel operator is adjusted according to the effect of the reconstructed image of the midpalatal suture in combination with an expert opinion. That is, the values of the elements of the matrices in the formula (3) are adjusted, and fusion and optimization of images are performed again. After a plurality of rounds of fusion and optimization, the optimal Sobel operator is selected according to the optimization so that the best fusion effect can be achieved. Finally, the problems of boundary blurring, unclear texture, and the like due to fusion are solved, and the purpose of image optimization is achieved. As shown in FIG. 4, a schematic diagram of fusion effects produced by a plurality of fusion approaches is shown. As can be seen, the fusion effect of the fusion approach in the present example is excellent.

Specifically, after obtaining the reconstructed image of the midpalatal suture, the method for reconstruction in the present example further includes the following steps:
(1) analyze a texture feature of the reconstructed image of the midpalatal suture to adjust a weight for the Sobel operator, thereby obtaining an optimized Sobel operator;
(2) perform merging fusion on all local images of the midpalatal suture to obtain a new reconstructed image of the midpalatal suture, where the optimized Sobel operator is utilized for optimization during the merging fusion;
(3) determine whether a maximum number of iterations is reached;
(4) if yes, determine an optimal Sobel operator based on optimization of all reconstructed images, where the reconstructed images include the reconstructed image of the midpalatal suture and the new reconstructed image of the midpalatal suture; and
(5) if no, take new reconstructed image of the midpalatal suture as the reconstructed image of the midpalatal suture in next loop and the optimized Sobel operator as the Sobel operator in next loop, and return to the step of analyzing a texture feature of the reconstructed image of the midpalatal suture.

The method for reconstructing the image of the midpalatal suture in the present example is mainly included three parts: CBCT file preprocessing, image fusion based on the Sobel operator, and analysis of a processing result of the midpalatal suture. After the CBCT file is obtained, data preprocessing is firstly performed thereon to process the CBCT filter into the three-dimensional gray-level matrix. The three-dimensional gray-level matrix is then processed into axial layered images, and the midpalatal suture region is cut to obtain the local images of the midpalatal suture. Finally, the local images of the midpalatal suture are normalized. After the data preprocessing is completed, merging fusion is performed on the local images of the midpalatal suture. A fusion parameter and weight may be calculated before the fusion each time. The fused image is optimized with the Sobel operator after the completion of each round of fusion. Finally, the fused image of the plurality of local images of the midpalatal suture is obtained, which is approximate to a projection of the overall morphological profile of the midpalatal suture on an axial plane. The method for reconstruction can be utilized to detect the development of a maxillary width of a teenager and evaluate the maxillary development potential of the teenager by extracting the images from the CBCT file of the midpalatal suture of the maxilla, and fusing and optimizing the images. The method for reconstruction can bring the following benefits:

(1) It is not only difficult but also subjective and abstract for human eyes to directly analyze the overall profile of the midpalatal suture in a multi-layer CBCT file, and it is impossible for one to intuitively feel the overall profile of the midpalatal suture region and hereby judge the development of the maxillary of a teenager. The method for reconstruction in the present disclosure enables direct observation of the projection of the overall morphological profile of the midpalatal suture in one image.

(2) In the present example, a CT image of the midpalatal suture region can be reconstructed by directly using a conventional CBCT file without extra CBCT examination at a particular angle. The difficulty of promotion and extensive use is greatly reduced. The method of the present disclosure is less affected by the camber of the midpalatal suture region of a patient in the process of reconstructing the midpalatal suture image, is universally applicable to people having large and small midpalatal suture cambers, and has better universality than other methods.

Compared with the traditional approach of acquiring the morphology of the midpalatal suture by obliquely photographing, the method for reconstructing the image of the midpalatal suture based on the Sobel operator technique used in the present example is to directly perform fusion and reconstruction on the midpalatal suture regions contained in the conventional multi-layer CBCT images and to restore the projection of the overall morphological profile of the midpalatal suture region on a plane. This process has no requirement on the photographing angle. Moreover, for a patient having a large midpalatal suture camber, the overall morphological profile of the midpalatal suture thereof can be displayed in one image. The application of the present example is time-saving and labor-saving for a doctor and enables more intuitive acquisition of the overall morphological profile of the midpalatal suture.

### Example 2

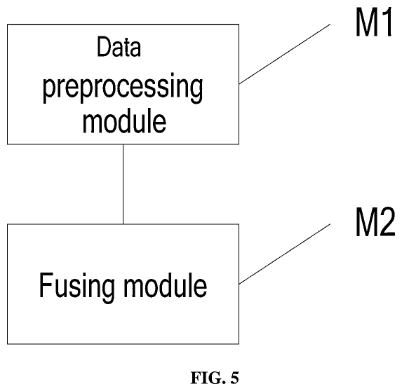

The present example is intended to provide a system for reconstructing an image of a midpalatal suture based on a Sobel operator. As shown in FIG. 5, the system includes:
a data preprocessing module M1 configured to perform data preprocessing on a CBCT file of a midpalatal suture of a maxilla to obtain a plurality of local images of the midpalatal suture; and
a fusing module M2 configured to perform merging fusion on the plurality of local images of the midpalatal suture to obtain a reconstructed image of the midpalatal suture, where a Sobel operator is utilized for optimization during the merging fusion.

The fusing module may include:
a combining unit configured to combine the plurality of local images of the midpalatal suture in pairs to obtain a plurality of image pairs to be fused, where pixels of the local images of the midpalatal suture included in each image pair to be fused correspond to one another one to one, and two pixels in the one-to-one correspondence are denoted as a pixel pair;
a fusing unit configured to, for each image pair to be fused, calculate a fusion weight for each pixel pair, and perform merging fusion on the image pair to be fused based on the fusion weight for each pixel pair to obtain a fused image;
an optimizing unit configured to optimize each fused image with the Sobel operator to obtain an optimized image;
a determining unit configured to determine whether a number of the optimized images is 1;
a reconstructing unit configured to, if yes, take the optimized image as the reconstructed image of the midpalatal suture; and
a returning unit configured to, if no, take the optimized images as the local images of the midpalatal suture in next loop, and return to the step of combining the plurality of local images of the midpalatal suture in pairs.

The description of each example in this specification focuses on a difference of the example from other embodiments. The same or similar parts of these examples may refer to each other. Since the system disclosed in an embodiment corresponds to the method disclosed in an embodiment, the description is relatively simple, and for related contents, references can be made to the description of the method.

Particular examples are used herein for illustration of principles and implementation modes of the present disclosure. The descriptions of the above embodiments are merely used for assisting in understanding the method of the present disclosure and its core ideas. In addition, those of ordinary skill in the art can make various modifications in terms of particular implementation modes and the scope of application in accordance with the ideas of the present disclosure. In conclusion, the content of the description shall not be construed as limitations to the present disclosure.

What is claimed is:

1. A method for reconstructing an image of a midpalatal suture based on a Sobel operator, comprising the following steps:
performing data preprocessing on a cone beam computed tomography (CBCT) file of a midpalatal suture of a maxilla to obtain a plurality of local images of the midpalatal suture; and
performing merging fusion on the plurality of local images of the midpalatal suture to obtain a reconstructed image of the midpalatal suture, wherein a Sobel operator is utilized for optimization during the merging fusion.

2. The method according to claim 1, wherein the performing data preprocessing on a CBCT file of a midpalatal suture of a maxilla to obtain a plurality of local images of the midpalatal suture specifically comprises the following steps:
reading the CBCT file of the midpalatal suture of the maxilla to obtain file information, wherein the file information comprises a resolution, a number of layers, a window width, and a window level of the CBCT file of the midpalatal suture of the maxilla;
transforming the CBCT file of the midpalatal suture of the maxilla into a three-dimensional gray-level matrix based on the file information, and transforming the three-dimensional gray-level matrix into a plurality of axial CT cross-sectional images; and
cutting each of the axial CT cross-sectional images containing midpalatal suture regions to obtain the plurality of local images of the midpalatal suture.

3. The method according to claim 2, wherein the transforming the CBCT file of the midpalatal suture of the maxilla into a three-dimensional gray-level matrix based on the file information specifically comprises the following steps:
determining a three-dimensional size of the three-dimensional gray-level matrix based on the resolution and the number of layers;
determining a gray level interval of the three-dimensional gray-level matrix based on the window width and the window level; and
transforming the CBCT file of the midpalatal suture of the maxilla into the three-dimensional gray-level matrix based on the three-dimensional size and the gray level interval.

4. The method according to claim 1, wherein the performing merging fusion on the plurality of local images of the midpalatal suture to obtain a reconstructed image of the midpalatal suture specifically comprises the following steps:
combining the plurality of local images of the midpalatal suture in pairs to obtain a plurality of image pairs to be fused, wherein pixels of the local images of the midpalatal suture comprised in each image pair to be fused are in one-to-one correspondence with each other, and two pixels in the one-to-one correspondence are denoted as a pixel pair;
for each image pair to be fused, calculating a fusion weight for each pixel pair, and performing merging fusion on the image pair to be fused based on the fusion weight for each pixel pair to obtain a fused image;
optimizing each fused image with the Sobel operator to obtain an optimized image;
determining whether a number of the optimized images is 1;
if yes, taking the optimized image as the reconstructed image of the midpalatal suture; and
if no, taking the optimized images as the local images of the midpalatal suture in next loop, and returning to the step of combining the plurality of local images of the midpalatal suture in pairs.

5. The method according to claim 4, wherein the calculating a fusion weight for each pixel pair specifically comprises the following steps:
calculating an overall average gray level and an adjustment factor based on gray level values of the plurality of local images of the midpalatal suture;
for each pixel pair, calculating an average value of gray level values of the image pair to be fused at the pixel pair to obtain an average gray level; and
calculating the fusion weight for the pixel pair based on the overall average gray level, the adjustment factor, and the average gray level.

6. The method according to claim 4, wherein the performing merging fusion on the image pair to be fused based on the fusion weight for each pixel pair to obtain a fused image specifically comprises the following steps:
for each pixel pair, calculating an average value of gray level values of the image pair to be fused at the pixel pair to obtain an average gray level; and
calculating a fused gray level value of the pixel pair based on the average gray level and the fusion weight to obtain the fused image.

7. The method according to claim 4, wherein the optimizing each fused image with the Sobel operator to obtain an optimized image specifically comprises the following step:
for each pixel of each fused image, calculating an optimized gray level value of the pixel based on the Sobel operator to obtain the optimized image.

8. The method according to claim 1, further comprising the following steps after obtaining the reconstructed image of the midpalatal suture:
analyzing a texture feature of the reconstructed image of the midpalatal suture to adjust a weight for the Sobel operator, thereby obtaining an optimized Sobel operator;
performing merging fusion on the plurality of local images of the midpalatal suture to obtain a new reconstructed image of the midpalatal suture, wherein the optimized Sobel operator is utilized for optimization during the merging fusion;
determining whether a maximum number of iterations is reached;
if yes, determining an optimal Sobel operator based on optimization of all reconstructed images, wherein the reconstructed images comprise the reconstructed image of the midpalatal suture and the new reconstructed image of the midpalatal suture; and
if no, taking the new reconstructed image of the midpalatal suture as the reconstructed image of the midpalatal suture in next loop and the optimized Sobel operator as the Sobel operator in next loop, and returning to the step of analyzing a texture feature of the reconstructed image of the midpalatal suture.

9. A system for reconstructing an image of a midpalatal suture based on a Sobel operator, comprising:
a data preprocessing module configured to perform data preprocessing on a CBCT file of a midpalatal suture of a maxilla to obtain a plurality of local images of the midpalatal suture; and
a fusing module configured to perform merging fusion on the plurality of local images of the midpalatal suture to obtain a reconstructed image of the midpalatal suture, wherein a Sobel operator is utilized for optimization during the merging fusion.

10. The system according to claim 9, wherein the fusing module specifically comprises:
a combining unit configured to combine the plurality of local images of the midpalatal suture in pairs to obtain a plurality of image pairs to be fused, wherein pixels of the local images of the midpalatal suture comprised in each image pair to be fused correspond to one another one to one, and two pixels in the one-to-one correspondence are denoted as a pixel pair;
a fusing unit configured to, for each image pair to be fused, calculate a fusion weight for each pixel pair, and perform merging fusion on the image pair to be fused based on the fusion weight for each pixel pair to obtain a fused image;
an optimizing unit configured to optimize each fused image with the Sobel operator to obtain an optimized image;
a determining unit configured to determine whether a number of the optimized images is 1;
a reconstructing unit configured to, if yes, take the optimized image as the reconstructed image of the midpalatal suture; and
a returning unit configured to, if no, take the optimized images as the local images of the midpalatal suture in next loop, and return to the step of combining the plurality of local images of the midpalatal suture in pairs.
